SEQUENCE LISTING (9 sequences)

SEQ ID NO: 1 - DNA, 38 bp, Artificial Sequence
  Description of Artificial Sequence Synthetic primer
aaagacttagacttctcagtgaaccagtaccgtgaggg

SEQ ID NO: 2 - DNA, 20 bp, Artificial Sequence
  Description of Artificial Sequence Synthetic primer
cgttacatcttccgcgcagg

SEQ ID NO: 3 - DNA, 15 bp, Artificial Sequence
  Description of Artificial Sequence Synthetic primer
gacttagacttctca

SEQ ID NO: 4 - DNA, 10 bp, Artificial Sequence
  Description of Artificial Sequence Synthetic primer
ttgctgggcg

SEQ ID NO: 5 - DNA, 11 bp, Artificial Sequence
  Description of Artificial Sequence Synthetic primer
gtggggtccat

SEQ ID NO: 6 - DNA, 12 bp, Artificial Sequence
  Description of Artificial Sequence Synthetic primer
atggggtgactg

SEQ ID NO: 7 - DNA, 10 bp, Artificial Sequence
  Description of Artificial Sequence Synthetic primer
cccactcccg

SEQ ID NO: 8 - DNA, 12 bp, Artificial Sequence
  Description of Artificial Sequence Synthetic primer
cctacaagtgcc

SEQ ID NO: 9 - DNA, 11 bp, Artificial Sequence
  Description of Artificial Sequence Synthetic primer
cctcggacgaa

(19) United States
(12) Patent Application Publication
(10) Pub. No.: US 2010/0273159 A1
(43) Pub. Date: Oct. 28, 2010

(54) Nested Multiplex Amplification Method for Identification of Multiple Biological Entities

(75) Inventors: Francisco Melo, Santiago (CL); Rodrigo Malig, Santiago (CL); Gaelle Lehouque, Santiago (CL); Denis Berndt, Santiago (CL)

Correspondence Address: MILLEN, WHITE, ZELANO & BRANIGAN, P.C., 2200 CLARENDON BLVD., SUITE 1400, ARLINGTON, VA, 22201, US

(73) Assignee: TAAG-GENETICS S.A., Santiago (CL)

(21) Appl. No.: 12/664,576

(22) PCT Filed: Jun. 20, 2007
(86) PCT No.: PCT/IB2007/001660

§ 371 Date: Jun. 14, 2010

(51) Int. Cl. C12Q 1/68; G01N 27/26

(52) U.S. Cl. 435/6; 204/452; 204/461

(57) ABSTRACT

The present invention provides a novel molecular method for the simultaneous identification and semi-quantification of multiple targeted biological entities from amongst a plurality. This invention discloses a method based on a multiplex nested amplification reaction in a single closed tube. The first amplification reaction relies on a set of large oligonucleotides for the amplification of common loci in all the targeted biological entities. The second nested amplification reaction relies on a set of short oligonucleotide primers that amplifies specific nucleotide sequences from all the amplicons previously produced in the first amplification reaction and generates an amplified product pattern capable of identifying each targeted biological entity. This method offers fast and accurate simultaneous identification of many targeted biological entities in any sample.

### FIELD OF THE INVENTION

The present invention relates to the field of bioinformatics and molecular biology and leads to the identification of multiple targeted biological entities from amongst a plurality using a multiplex nested amplification reaction in a single closed tube.

### BACKGROUND OF THE INVENTION

Rapid and accurate identification of biological entities—any biological agent or organism—is crucial in a variety of industrial, medical, environmental and research applications.

Three classes of identification tests are commonly used in the detection of biological agents: i) immunoabsorbant-based tests; ii) cell culture methods; and iii) molecular biology tests.

The use of molecular biology assays has grown significantly because they have several advantages over non-DNA based approaches. For instance, the molecular biology tests are more sensitive, much faster and more accurate than the traditional approaches.

Molecular biology tests commonly used comprise: i) microarrays; ii) other DNA probe-based; iii) polymerase chain reaction (PCR); iv) multiplex PCR; and v) nested multiplex PCR.

Microarrays-based strategies have been developed for the detection and identification of multiple bacteria in biological samples (Rudi, K. et al. 2002. Appl. Environ. Microbiol. 68(3): 1146-1156; Palmer et al. 2006. Nucleic Acids Res. 34(1): e5). Although these approaches allow a high-throughput performance, they have several drawbacks, which include: i) Need of a time-consuming hybridization step of about 16 hours (which limits their application in some problems of clinical diagnosis). ii) High set-up costs (which are unsuitable when a relatively small number of samples need to be studied, such as some clinical research applications). iii) Limited discrimination capacity (this methodology does not allow to discriminate highly-related organisms such as different strains of the same species). iv) Requirement of high-cost and specialized laboratory equipment, and v) need of specialized technical skills for performing the tests.

Other DNA probe-based tests have been developed for the detection and identification of some known bacterial pathogens (PCT patent application serial No. WO 93/03186; Quere, F. et al. 1997. Journal of Applied Microbiology. 82: 783-790). However, these strategies are based on the amplification of highly conserved genes, such us the 16S rRNA gene, followed by hybridization with species-specific oligonucleotides. These approaches are time-consuming because of the hybridization step. In addition their discrimination ability is poor (U.S. Pat. No. 6,994,965), not allowing the identification of highly related organisms.

Most of molecular biology tests generally use any one of a number of variations on the PCR, which can detect genetic material of an organism from biological samples suspected of containing the targeted biological agent.

Traditional single locus PCR tests provide fast results, but because they make use of long oligonucleotide primers (˜20 nts. long), they are not able to identify highly related biological entities (poor discrimination ability). Two hybridized strands with partially non-complementary sequences can have a sufficiently high binding energy when they contain complementary regions of sufficient lengths (U.S. Pat. No. 6,994,965), producing unspecific amplification products. Therefore, this methodology has low discrimination ability and it can become difficult, if not impossible, to distinguish among highly related targets (Settanni, L. et al. 2005. Appl. Environ. Microbiol. 71, 3049-3059; Settanni, L. 2007, Journal of Microbiological Methods 69 1-22).

Multiplex PCR allows the amplification of target sequences from multiple organisms in one reaction using multiple sets of locus specific primers. Therefore, multiplex PCR is suited to multiple detection. However, multiplex PCR methods have limitations. Besides of the lack of specifity, combining multiple target loci in one reaction may introduce incompatibility between various primer sets which results in poor amplification or inhibition of some amplification reactions. Therefore the Multiplex PCR approaches only can detect a low number of biological entities in a single reaction, and it does not allow identification of highly related organisms.

Nested multiplex PCR approaches are frequently the adopted strategy when the speed and sensitivity of organism detection are crucial, as for example in clinical diagnosis (McManus, P. S., and A. L. Jones, 1995. Phytopathology 85:618-623). However, standard multiplex nested-PCR methods are labour intensive and have a high false positive rate because of cross-contamination caused by the manipulation of previously amplified material, thus making this approach too risky for routine analysis (Llop, P. et al. 2000. Appl Environ Microbiol. 66(5): 2071-2078).

To avoid the manipulation of the amplified material between the first and second round of amplification, a method based on the multiplex nested amplification reaction in a single tube has been described (Olmos, A. et al. 1999. Nucleic Acid Res. 27(6): 1564-1565). However, this method has two serious disadvantages. First, the PCR products need to be vortexed and centrifuged between both amplification steps, thus it becomes more difficult to automate the process. Second, the same drawbacks described above for multiplex amplification-based methods are present (low simultaneous multiplexing capacity and poor discrimination ability).

Another approach for nested multiplex amplification of nucleic acids is called Templex (J. Han, PCT number WO 2005/038039 A2). This technology is based on three serial amplification stages and the amplification products are detected through target-specific probes differentially labelled. The Templex approach has two main disadvantages. One is that several distinct oligonucleotide sequences are required for a single target. For example, this technology requires 4n+2 different primers to achieve the successful amplification of n targets. The second limitation of the Templex method is that it is not able to discriminate between highly related targets, because it relies on using four ˜20 nucleotide long target-specific sequences. These nucleotides sequences must have several mismatches for the non-targeted organisms to avoid the detection of false positives. Even for traditional multiplex PCR, this is very difficult to achieve among related bacteria, (Settanni, L. et al. 2005. Appl. Environ. Microbiol. 71, 3049-3059; Settanni, L. 2007, Journal of Microbiological Methods 69 1-22). Additionally, the use of a probe-hybridization step not only limits its discrimination ability, but it also increases the time required to perform the analysis. These restrictions are well-known to those skilled in the art (U.S. Pat. No. 6,994,965).

As it was mentioned above, all the existing techniques for the detection of organisms have several drawbacks. In view of this, a great need for efficient methods to detect and identify several targeted biological entities from amongst a plurality remains. Herein a quick and accurate DNA-based nested multiplex detection method is disclosed, which offers many advantages over the prior art. Briefly nucleic acid samples are obtained from samples suspected of containing the interest bioagent(s); the nucleic acid may be DNA or RNA (either positive strand or negative strand) or a combination thereof. A nested multiplex amplification reaction at two temperatures in a single closed tube is used to amplify pre-determined target sequences from the nucleic acid. The amplification pattern produced allows an easy and simultaneous detection of the targeted biological entities. Using the method disclosed herein, the detection of the targeted biological entities can be made in as little as 3 hours.

### SUMMARY OF THE INVENTION

The present invention provides a method to simultaneously detect and semi-quantify several targeted biological entities from amongst a plurality in a sample. The method consists of a multiplex nested amplification reaction carried out in a single closed tube, characterized by:
1. A first amplification reaction in which a set of long oligonucleotide primers (larger than 15 nucleotides) is used to amplify common loci in all the targeted biological entities.
2. A second amplification reaction led by short oligonucleotide primers, directed to sequences of less than 16 nucleotides long, that amplifies specific nucleotide sequences from all the amplicons previously produced in the first amplification reaction and generates an amplified product pattern capable of identifying each targeted biological entity.

The short length of the oligonucleotide primers (in the range of 4 to 15 nucleotides) used in the second amplification reaction provides a remarkable discrimination capacity (ie. sequences differing in only one nucleotide can be resolved). In addition to this, the reduced number of oligonucleotide primers in the reaction tube allows an exceptionally high multiplexing capacity.

The method described herein uses two different annealing temperatures for each step of the nested amplification, thus allowing that the whole reaction is carried out in one single and close tube. This feature reduces cross-contamination risks, costs and time as compared to standard multiplex nested-PCR protocols. The method can also be easily automated.

The method described in the present invention can be used, among other applications, for the simultaneous identification and semi-quantification of several organisms amongst a plurality in a sample in one single high throughput test. More specifically, the method is useful for the fast and accurate identification and quantification of viral, bacterial and eukaryotes in clinical or industrial samples.

### BRIEF DESCRIPTION OF THE DRAWINGS

FIG. 1 illustrates one embodiment of the method described in the present invention.

FIG. 2 illustrates the results obtained by agarose electrophoresis for example 1.

FIG. 3 illustrates the results obtained by agarose electrophoresis for example 2.

FIG. 4 illustrates the results obtained by capillary electrophoresis for example 3.

### DETAILED DESCRIPTION OF THE INVENTION

Prior to describe this invention, it may be helpful to define a set of terms that will be used hereinafter.

As used herein, a biological entity, biological agent or bioagent refers to an individual or a mixture of individuals such as viruses, prokaryotes or eukaryotes.

As used herein, targeted biological entities comprise the taxon of the biological entities (ie. species, strains, varieties, isolates, etc.) which presence or absence in a sample is being evaluated. The non-targeted biological entities correspond to bioagents likely to be present in the sample but not relevant for the analysis.

As used interchangeably in this disclosure, the terms “sample” or “biological sample” include any specimen or culture of biological and environmental samples or nucleic acid isolated therefrom. Biological samples may be animal, including human, fluid, such as blood or urine, solid or tissue, alternatively food and feed products and ingredients such as dairy items, vegetables, meat and meat by-products. Environmental samples may include material such as surface matter, soil, water, industrial samples and waste, for example samples obtained from sewage plant, as well as samples obtained from food and dairy processing instruments, apparatus, equipment, utensils, disposable and non-disposable items.

As used herein, the term “discrimination ability” refers to the capacity to identify and differentiate highly related biological entities. On the other hand, the term “poor discrimination ability” is used herein for the same situation, but when such discrimination is not achieved.

As used interchangeably in this disclosure, the terms “nucleic acid molecule (s)”, “oligonucleotide (s)”, and “polynucleotide (s)” include RNA or DNA (either single or double stranded, coding, complementary or antisense), or RNA/DNA hybrid sequences of more than one nucleotide in either single chain or duplex form (although each of the above type of molecules may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (ie. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide.

The term “nucleotide” is also used herein to encompass “modified nucleotides” which comprise at least one modification such as (a) an alternative linking group, (b) an analogous form of purine, (c) an analogous form of pyrimidine, or (d) an analogous sugar. Any polynucleotide sequences optimally designed to be used by the method described in this invention may be prepared by any known procedure, including synthetic, recombinant, ex vivo generation, or a combination thereof, as well as utilizing any purification methods known in the art.

The various components of the present invention are described in greater detail below. It should be appreciated that while certain embodiments are discussed in regard to these components, other methods known in the art for accomplishing the same ends should be considered within the scope of the present disclosure. In addition, various embodiments of the present invention may use different methods for carrying out the steps described below, depending on the purpose of the method.

The method described herein is capable of simultaneously detecting multiple targeted biological entities from amongst a plurality in a sample. The method is useful in a wide variety of fields, including, but not restricted to, clinical diagnostics, microbiological control, traceability and environmental testing.

The present method can be used to detect and identify any combination of biological agents, including virus, archaeas, bacteria, fungi, plants, animals, and any combination thereof. It allows the identification of highly related biological entities, such as varieties and strains, from both isolates or complex mixtures. In addition to this, it is able to identify bioagents belonging to lower specific taxonomic groups, for instance phylum, class, order, family, genus and species.

The present invention described herein can be used to simultaneously detect and semi-quantify any combination of DNA or cDNA obtained from a previous step of reverse transcription (RT) with or without an additional step of PCR amplification.

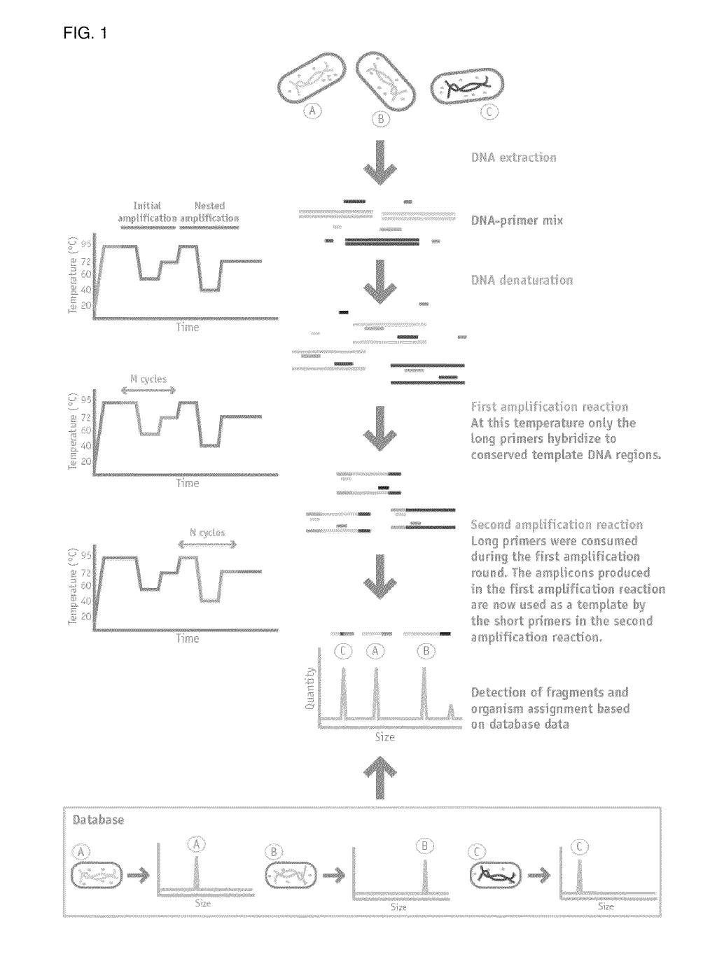

The method is based on a nested multiplex amplification reaction in a single closed tube where the first amplification reaction is led by a set of long oligonucleotide primers used to amplify common loci in all the targeted biological entities and the second amplification reaction is led by short oligonucleotide primers used to hybridize the products from the first amplification reaction, as shown in FIG. 1.

The oligonucleotide primers are designed by optimizing several restraints, which include molecular interactions, melting temperatures and target specificity. This can be achieved by the integration of ordinary bioinformatics software. Any skilled in the art can create the necessary software to accomplish the optimized design of the oligonucleotides primers. For the examples of the present invention the oligonucleotides were designed by our own custom software.

The oligonucleotide primers leading the first amplification round, hereby defined as “long primers”, were designed by simultaneously optimizing the following restraints:

a) the successful amplification of common loci from all the targeted biological entities with a set of long primer pairs (equal or higher than 16 nucleotides);

b) all long primers must have an annealing temperature difference among them of less than 5° C.;

c) the annealing temperature must be in the range 50 to 54° C., preferably of 55 to 60° C. and most preferably of 61 to 75° C., and;

d) the non-amplification of the non-targeted bioagents likely to be present in the analyzed biological sample. The careful design of the long oligonucleotide primers contributes to insure a high specificity and to maximize the multiplexing capacity.

The embodiment illustrated in FIG. 1 shows the use of a single pair of long primers to amplify a common locus in all targeted biological entities. Alternatively more than one primer pair may be used if desired.

If the targeted biological entities are from distinct genus or distantly species, the template nucleotide sequences or loci used to design the long primer pair(s) should have a low inter-species variation rate. By the term “low inter-species variation rate”, it is meant that the template nucleotide sequences share a sequence identity of 70-99%, more preferably 80-99% and most preferably 90-99%.

If the targeted biological entities are closely-related bioagents, such as distinct strains or varieties of the same species, the design of the long primer pair(s) should be directed to amplify nucleotide sequences that exhibit a high variation rate, for instance those genomic regions with neutral or without evolutionary selective pressure.

In accordance with the invention, specific nucleotides of the long primers could be substituted by non standard bases, such as inosine, locked nucleic acid molecules, uridine, 2,6-diaminopurine, propyne C, or propyne T.

The invention provides a further embodiment which relates to adding a 5′ extension tail to one of the long primers of each selected pair. This helps to reduce the total number of primers required for the second round of amplification, thus increasing the potential multiplexing level of the technique. The nucleotide tail should consist of a sequence absent in the genomes of the targeted biological entities, to avoid amplification of unspecific products.

The second amplification step uses short oligonucleotide primers that amplify specific nucleotide sequences from all the amplicons previously produced in the first amplification reaction, hereby defined as “short primers”. All These short primers were designed by simultaneously optimizing the following restraints:

a) all short primers must have a length in the range of 4 to 15 nucleotides, preferably 4 to 14 nucleotides, and most preferably 4 to 13 nucleotides;

b) all short primers must not contain low complexity sequences such as mono, di, tri or tetra nucleotide repeats;

c) the short primers must not have stable inter molecular interactions between them;

d) all short primers must have an annealing temperature difference among them of less than 5° C.;

e) all short primers must have an annealing temperature lower than that of the long primers;

f) the annealing temperature difference between the long and short primers must be in the range 10 to 50° C., preferably of 10 to 45° C. and most preferably of 15 to 40° C., and;

g) the second amplification reaction led by the short primers must generate a unique product size pattern for each targeted biological entity.

In a most preferred embodiment two additional restraints are optimized allowing the identification of N targeted biological entities with just N+1 short primers:

a) a single short primer (named SST) must hybridize to one of the long primers or to its 5′ tail sequence, and;

b) each of the remaining short primers (named RSPs) must specifically hybridize to a single amplicon of the first amplification reaction. In addition to this, the RSPs and the SST must hybridize to opposite strands in the amplicons.

This preferred embodiment is illustrated FIG. 1. Although the embodiment illustrated in FIG. 1 shows the use of N+1 short primers to identify N targeted biological entities, less than N+1 short primers may be used if they are capable of identifying all the targeted biological entities.

According to previous art (Caetano-Anolles G. 1993. PCR Methods Appl. 3(2):85-94) and to our own experimental data, a single mismatch within the first five nucleotides from the 3′ end of the short primers and their templates avoids amplification. In the present invention, this characteristic must be considered when designing the short primers, as it is essential for the identification of highly related biological entities.

In accordance with the invention, specific nucleotides of the short primers could be substituted by non standard bases, such as inosine, uridine, 2,6-diaminopurine, propyne C, or propyne T.

In an alternate embodiment, the short primers may have a secondary structure (e.g., a hairpin at the 5′ end) to recognize even shorter template sequences (Caetano-Anolles, 1993. PCR Methods Appl. 3(2):85-94).

In accordance with the invention, any short primer could also be labeled, for example with a fluorescent dye. This strategy has several advantages, which are: 1) high-throughput analysis is facilitated; 2) the sensitivity for signal detection is increased; 3) semi-quantification of the amount of each amplification product by comparison with internal standards is possible; and 4) more complex patterns can be generated and resolved by the simultaneous utilization of multiple channels.

This strategy of using the short oligonucleotides in a second nested amplification reaction, along with the previously described scheme for designing the long primers, contributes to reduce even more the false positive rate, as compared to previous art.

The present invention shows an extraordinary high multiplexing capacity that results from:

As illustrated in FIG. 1, for most applications, the total number of oligonucleotide primers used in the method of the present invention is just 3+N (2 long primers plus N+1 short primers), where N is the number of targeted biological entities. Therefore, compared to prior art the method described here uses a much lower number of oligonucleotide primers in solution, consequently reducing the total amount of inter-molecular interactions and thus maximizing the multiplexing capacity.

The small length of the short primers reduces even more the intra e inter-molecular interactions.

There is no PCR-based method in the prior art that allows the identification of N biological entities with as few oligonucleotide primers as the present invention. Thus, our method has a higher multiplexing capacity than other PCR-based methods described in the previous art.

This higher multiplexing capacity allows the identification of a greater number of biological entities in a sample. This feature greatly reduces time and costs currently required for the identification of multiple biological entities in clinical and industrial fields.

The use of short primers (4-15 nts long) in the second amplification reaction allows the detection of single nucleotide sequence variations between the targeted biological entities. Due to the small size of short primers, a single mismatch within the first five nucleotides from the 3′ end of the short primers and their templates avoids amplification (Caetano-Anolles G. 1993. PCR Methods Appl. 3(2):85-94.).

There is no PCR-based methods in the prior art that allows the simultaneously identification of highly related biological entities, with the level of specificity shown in the present invention. Thus, our method has the higher specificity, or discrimination ability, of all PCR-based methods described in the previous art.

This feature makes the method of this invention ideal to detect and identify closely-related species, such as distinct strains or varieties of the same species amongst a plurality of related bioagents. This is difficult, if not impossible, to achieve when using the typical PCR-based methods.

An important embodiment of this invention is the use of a single closed tube for the whole multiplex nested amplification reaction. This allows to simultaneously detect and semi-quantify many targeted biological entities with high sensitivity and specificity, which reduces cross-contamination risks, costs, time and it allows an easy automation.

In accordance with the invention, and to achieve two successive multiplex amplification reactions in the same closed tube, two criteria need to be accomplished:

a) the annealing temperatures must have a difference between both rounds of amplification of about 10 to 50° C., preferably of about 10 to 45° C., and most preferably of about 15 to 40° C., and;

b) the short-primer/long-primer concentration ratio must be within a fixed range, with the concentration of large primers much lower than the concentration of the short primers. The concentration of large primers should be from about 1 to about 20,000, preferably from about 10 to about 10,000 and most preferably from about 100 to about 5,000.

During the first amplification reaction, the high difference in the annealing temperature between long and short primers, and consequently between the two amplification steps, significantly reduces their interaction and avoids the hybridization of the short primers with any other nucleic acid molecules, especially with the template DNA of the first amplification reaction.

Because the long primers are used at a low concentration, they are totally consumed during the first amplification reaction; hence in the second amplification no interaction between long and short primers will take place.

The difference in annealing temperature of the two steps of the nested amplification allows that the whole reaction is carried out in a single close tube. Therefore, the amplification reaction is more efficient (lower contamination risk and easier automation) and with lower molecular interactions than traditional multiplex PCR methods.

The scope of this invention is not limited to the use of amplification by PCR-based methods, but it rather includes the use of any rapid nucleic acid amplification method employed to increase rapidity and sensitivity of the tests.

In an alternate embodiment, the method could be carried out using isothermal amplification procedures. In these cases, the method will be performed using two separate reactions or a compartmentalized tube. In any case, the first amplification reaction should be carried out at a high temperature while the second one at a low temperature.

Isothermal amplification procedures includes, but are not limited to transcription-mediated amplification (TMA), self-sustained sequence replication (3SR), nucleic acid sequence-based amplification (NASBA), strand displacement amplification (SDA), branched DNA (bDNA) and cycling probe technology (CPT) (Lee et al., 1997. Eaton Publishing, Boston, Mass.; Persing et al., 1993. American Society for Microbiology, Washington, D.C.).

As mentioned previously, the method described herein can be used for the simultaneous semi-quantification of the analyzed biological entities. To accomplish this, an internal standard of a previously quantified nucleic acid extract must be used. In addition to this, a reduced number of cycles (less than 30) for each step of the nested amplification are required for achieving the exponential amplification of the target sequences.

In the present invention, the sample may be unprocessed raw material or material prepared by using any of the standard methods well-known in the art for isolating and preparing nucleic acids for PCR amplification. Samples may be obtained from any organism or source from which DNA or RNA may be derived. In one embodiment, the target nucleic acid is a single-stranded RNA that must be first reverse-transcribed and copied into double-stranded DNA.

For the present invention any method for oligonucleotide detection and identification could be used to generate the amplified product pattern necessary for the identification of the targeted biological entities. In one embodiment of the method described herein, the size differences between the amplicons produced in the second amplification reaction allows the easy and simultaneous detection of the targeted biological entities.

In a preferred embodiment, capillary electrophoresis (CE) equipment may be used to separate and detect the amplification products produced by the method disclosed in this invention.

In a most preferred embodiment, micro-channel fluidic devices are used to separate and detect the amplification products produced by the multiplex nested amplification reaction. Micro-channel fluidics has a high resolution power (5 by in a range of 25-100 nts and 5% of resolution from 100-700 bp), allows automated quantification of each nucleotide fragment against internal standards, the detection takes only 30 minutes and has high-throughput performance allowing the simultaneous analysis of several different samples.

The embodiment illustrated in FIG. 1 uses amplicon size differences to identify all the targeted biological entities, however other detection methods may also be used, including, but not limited to, detection of fluorescence after amplification (ie. TaqMan™ system from Perkin Elmer or Amplisensor™ from Biotronics) or detection performed by solid support or liquid hybridization using nucleotide probes hybridizing to at least one amplification product. The oligonucleotide probes may be labeled with biotin or with digoxigenin or with any other reporter molecule.

Semi-quantitative or quantitative amplification procedures, as well as semi or full automation of the method are also under the scope of the invention.

As will be apparent to those skilled in the art, in light of the foregoing disclosure, many modifications, alterations and substitutions are possible in the practice of this invention without departing from the spirit or the scope thereof. The scope of the invention should, therefore, be not determined with reference to the above description, but should instead be determined with reference to the appended claims, along with the full scope of equivalents to which such claims are entitled.

### EXAMPLES

The following examples provide an illustration of the usefulness of the invention which is nevertheless not limited to these examples. The details of the oligonucleotide primer mix used in all examples, as well as the predicted amplicon sizes, are specified in Table 1.

### Example 1

The method described herein was used to identify each of the following bacteria: Burkholderia vietnamiensis strain G4, Burkholderia xenovorans strain LB400, Escherichia coli strain ATCC 25922, Pseudomonas aeruginosa strain ATCC 27853, Pseudomonas putida strain KT2440 and Vibrio cholerae strain 0395.

Nested multiplex PCR in a single closed tube was performed from a sample consisting of a dilution of a bacterial colony or an overnight liquid bacterial culture. A single colony or 1 ul of liquid culture was resuspended in 150 uL or 300 uL of nuclease-free water.

1 to 4 uL were used for PCR amplification in a total volume of 15 uL, containing 20 mM Tris-HCl (pH 8.4), 50 mM KCl, 2 mM MgCl2, 200 mM of each deoxynucleoside triphosphate, 0.01 pmol of each long primer, 1 pmol of each short primer and 1.2 U of Platinum Taq DNA polymerase (Invitrogen).

The PCR amplifications were carried out in an Applied Biosystems 2720 thermal cycler as follows: after an initial denaturation step of 95° C. for 7 min, the first round of the nested PCR consisted in 27 cycles of 94° C. for 30 s, 62° C. for 30 s and 72° C. for 50 s. The second round consisted of 15 cycles of 94° C. for 30 s, 42° C. for 30 s, and 72° C. for 30 s and a final extension cycle of 5 min at 72° C. As a negative control, bacterial samples were substituted with nuclease-free water.

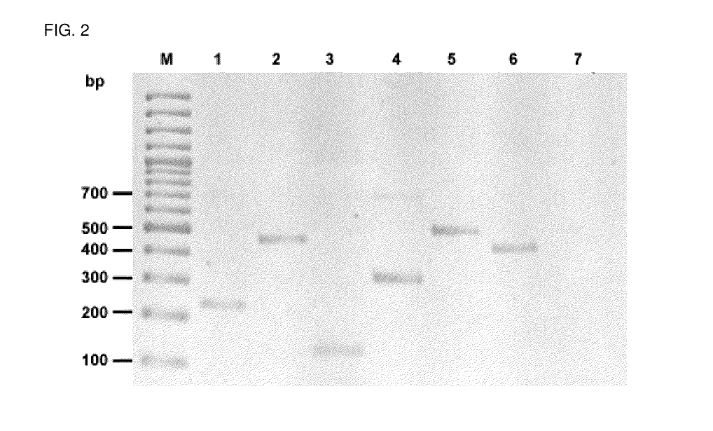

PCR amplification products were analyzed by standard agarose gel (2%) electrophoresis and visualized under UV at 254 nm after staining in a 0.5 μg/mL ethidium bromide solution, as illustrated in FIG. 2 (lane M: 100 by ladder, lane 1: Burkholderia vietnamiensis strain G4, lane 2: Burkholderia xenovorans strain LB400, lane 3: Escherichia coli strain ATCC 25922, lane 4: Pseudomonas aeruginosa strain ATCC 27853, lane 5: Pseudomonas putida strain KT2440, lane 6: Vibrio cholerae strain 0395 and lane 7 is the negative control).

As it is shown in FIG. 2, the method of the present invention achieves the specific amplification of each tested target, thus allowing the identification of multiple biological entities.

### Example 2

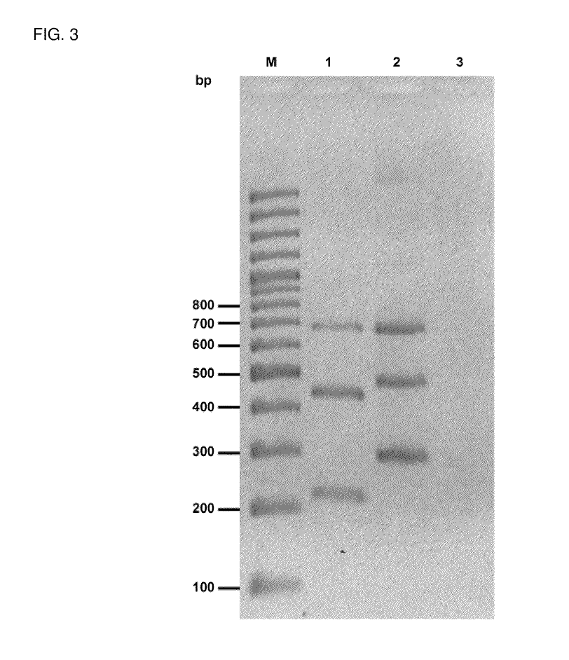

Same as example 1, except that amplification was performed on bacterial suspensions consisting of a mixture of 2 highly related bacteria. As it can be seen in FIG. 3, the method of the present invention achieves the specific amplification of each target, thus allowing the simultaneous identification of highly related biological entities from a binary mixture. In FIG. 3, lane M is 100 bp ladder, lane 1 is Burkholderia vietnamiensis strain G4 plus Burkholderia xenovorans strain LB400, lane 2 is Pseudomonas aeruginosa strain ATCC 27853 plus Pseudomonas putida strain KT2440 and lane 3 is the negative control).

### Example 3

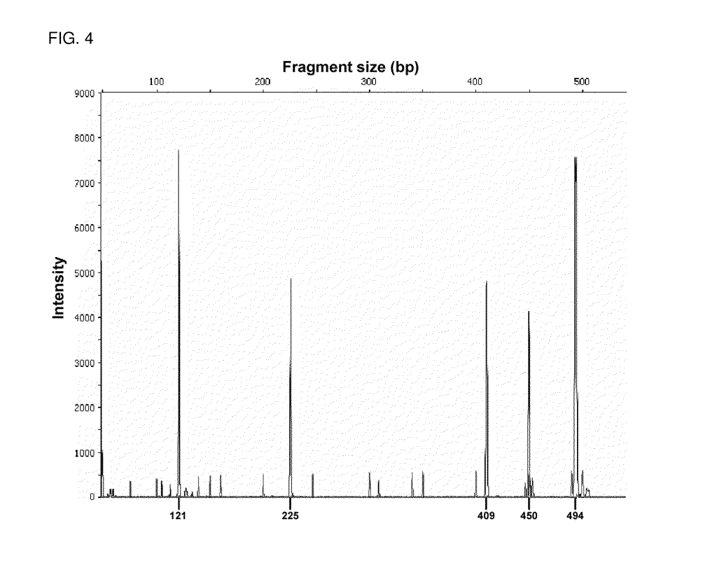

Same as example 2, except that amplification was performed on a bacterial suspension consisting of a mixture of 5 species of bacteria (Escherichia coli strain ATCC 25922, Burkholderia vietnamiensis strain G4, Vibrio cholerae strain O395, Burkholderia xenovorans strain LB400 and Pseudomonas putida strain KT2440). In this example the PCR amplification products were separated and detected using the capillary electrophoresis system ABI 3100. As it can be seen in FIG. 4, the method of the present invention achieves the specific amplification of each target, thus allowing the simultaneous identification of multiple biological entities from a complex mixture.

TABLE 1
Oligonucleotide primer sequences used in all the examples
of the present invention.

| | | | | Amplicon |
|---|---|---|---|---|
| | | Tm | | size |
| lD^{‡} | Oligonucleotide sequences^{†} | (° C.) | Target | (bp) ^{#} |
| LP1 | 5′- | 62. | Conserved 23S | 680 |
| | AAAGACTTAGACTTCTCAGTGAACCAGTA | 9 | rRNA sequence | |
| | CCGTGAGGG | | | |
| LP2 | 5′-CGTTACATCTTCCGCGCAGG | 64. | Conserved 23S | |
| | | 5 | rRNA sequence | |
| SST | 6FAM-5′-GACTTAGACTTCTCA | 44. | Subsequence of | |
| | | 4 | the 5′ tail of LP1 | |
| RSP | 5′-TTGCTGGGCG | 44. | *Burkholderia | 224 |
| 1 | | 3 | vietnamiensis | |
| | | | strain G4 | |
| RSP | 5′-GTGGGGTCCAT | 42. | *Burkholderia | 449 |
| 2 | | 5 | xenovorans strain | |
| | | | LB400 | |
| RSP | 5′-ATGGGGTGACTG | 44. | *Escherichia coli | 121 |
| 3 | | 5 | strain ATCC | |
| | | | 25922 | |
| RSP | 5′-CCCACTCCCG | 42. | *Pseudomonas | 305 |
| 4 | | 5 | aeruginosa strain | |
| | | | ATCC 27853 | |
| RSP | 5′-CCTACAAGTGCC | 43. | *Pseudomonas | 494 |
| 5 | | 3 | putida strain | |
| | | | KT2440 | |
| RSP | 5′-CCTCGGACGAA | 43. | *Vibrio cholerae | 408 |
| 6 | | 5 | strain O395 | |

^{‡}LP1 and LP2 lead the first amplification round. The remaining primers lead the second amplification round. SST is common for all second amplification reactions whereas the RSPs are species-specific.
^{†}The underlined sequence corresponds to a non-hybridizing 5′tail. 6FAM is a dye used to detect the amplification products by the capillary electrophoresis system ABI 3100.
*These are short species-specific 23S rRNA sequences found in the amplicons generated during the first amplification reaction led by the long primers LP1 and LP2.
^{#}These numbers represent the size of the amplicons produced by LP1-LP2 and by the combination of SST with each of the RSPs primers.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method to simultaneously identify and semi-quantify at least three targeted biological entities in a sample from amongst a plurality, wherein said method is a multiplex nested amplification reaction comprising:
a) A multiplex nested amplification with all the reagents in a homogeneous solution.
b) A first amplification reaction led by a set of long oligonucleotide primers, of at least 16 nucleotides long, is used to amplify common loci from all the targeted biological entities.
c) A second nested amplification reaction led by short oligonucleotide primers, directed to sequences of less than 16 nucleotides long, that amplifies specific nucleotide sequences from all the amplicons previously produced in the first amplification reaction and generates an amplified product pattern capable of identifying each targeted biological entity.

2. The method of claim 1, wherein said short oligonucleotide primers comprise oligonucleotide primers directed to nucleotide sequences from 4 to 15 nucleotides long, preferably from 4 to 14 nucleotides long, and most preferably from 4 to 13 nucleotides long.

3. The method of claim 2, wherein said short oligonucleotide primer(s) hybridize any nucleotide sequence present in the long primers.

4. The method of claim 1, wherein said method comprises a nested multiplex amplification reaction where both steps of amplification have an annealing temperature difference from about 10 to 50° C., preferably from about 10 to 45° C., and most preferably from about 15 to 40° C.

5. The method of claim 1, wherein said method comprises a nested multiplex amplification reaction where the concentration ratio of the oligonucleotide primers used in the second and first amplification reactions is about 1 to 20,000, preferably from about 10 to 10,000, and most preferably from about 100 to 5,000.

6. The method of claim 1, wherein said multiplex nested amplification reaction is carried out in a single closed tube.

7. The method of claim 1, wherein said multiplex nested amplification reaction comprises a multiplex nested non-isothermal amplification reaction.

8. The method of claim 7, wherein said multiplex nested non-isothermal amplification reaction further comprises a polymerase chain reaction.

9. The method of claim 1, wherein said multiplex nested amplification reaction comprises an isothermal amplification reaction.

10. The method of claim 9, wherein said multiplex nested isothermal amplification reaction comprises the following methods:
a) loop-mediated isothermal amplification (LAMP),
b) helicase-dependent amplification (HDA),
c) nucleic acid sequence-based amplification (NASBA),
d) strand displacement amplification (SDA),
e) transcription-based amplification system (TAS),

11. The method of claim 1, wherein said oligonucleotide primers are specific or degenerated oligonucleotide primers.

12. The method of claim 11, wherein said degenerated oligonucleotide primers comprise at least one nucleotide analogue, as for example: inosine, uridine, locked nucleic acid molecules, 2,6-diaminopurine, propyne C, or propyne T.

13. The method of claim 1, wherein said first amplification reaction is led by oligonucleotide primers having a 5′ nucleotide sequence capable of hybridizing the oligonucleotide primers used in the second amplification reaction.

14. The method of claim 1, wherein said first amplification reaction is led by oligonucleotide primers targeted to one o more loci.

15. The method of claim 1, wherein any of the said oligonucleotide primers is labelled with a molecule that can be used either to report a signal or as a capture agent.

16. The method of claim 15, wherein the labeled primer comprises a radioactive primer, a primer containing a fluorophore or the biotin molecule.

17. The method of claim 1, wherein said biological entities comprise DNA or cDNA from virus, prokaryotes or eukaryotes.

18. The method of claim 1, wherein the amplified product pattern is generated by a micro-channel fluidics system.

19. The method of claim 1, wherein the amplified product pattern is generated by capillary electrophoresis.

20. The method of claim 1, wherein the amplified product pattern is generated by a method that comprises any of the following:
a) high performance liquid chromatography (HPLC),
b) gel electrophoresis,
c) electrochemiluminescence,
d) immunochemically,
e) mass spectrometry, and
f) hybridization to oligonucleotides or probes, whether or not they are immobilized to a solid support.
